(19) United States
(12) Patent Application Publication
(10) Pub. No.: US 2004/0142334 A1
(43) Pub. Date: Jul. 22, 2004

(54) Diagnosis of diseases associated with angiogenesis

(76) Inventor: Oliver Schacht, Berlin (DE)

Correspondence Address: Kriegsman & Kriegsman, 665 Franklin Street, Framingham, MA, 01702, US

(21) Appl. No.: 10/433,793

(22) PCT Filed: Dec. 6, 2001
(86) PCT No.: PCT/EP01/14320

(30) Foreign Application Priority Data
Dec. 6, 2000 (DE) 10061338.1

(51) Int. Cl. C12Q 1/68; C07H 21/04; C07K 14/00

(52) U.S. Cl. 435/6; 530/350; 536/23.2

(57) ABSTRACT

The present invention concerns the chemically modified genomic sequences of genes associated with angiogenesis, oligonucleotides and/or PNA oligomers directed against the sequence for the detection of the cytosine methylation state of genes associated with angiogenesis as well as a method for determining genetic and/or epigenetic parameters of genes associated with angiogenesis.

### FIELD OF THE INVENTION

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome. In this regard, pathogenic states are also expressed by a modified methylation pattern of individual genes or of the genome.

The present invention concerns nucleic acids, oligonucleotides, PNA oligomers and a method for the diagnosis and/or therapy of diseases that are linked to genetic and/or epigenetic parameters of genes associated with angiogenesis and particularly their methylation state.

### PRIOR ART

Angiogenesis describes the process of the formation of new blood vessels. This process, which is also denoted neovascularization, normally occurs during embryogenesis and placental development, but also occurs in the adult organism and during wound healing. Stimulation by one or more known growth factors initiates angiogenesis; however, other factors that have not as yet been identified may also participate therein. Angiogenesis is controlled by the equilibrium between stimulators and inhibitors and is suppressed under normal physiological conditions. A displacement of this equilibrium plays an important role in the development of a number of diseases, whereby differences in this equilibrium vary in the different organs.

Pathogenic conditions, with which angiogenesis is linked, include, for example, eye diseases, proliferative retinopathy, neovascular glaucoma, solid tumors and diseases, which are associated with tissue inflammation, such as rheumatoid arthritis (Moses M A, Langer R. Inhibitors of angiogenesis, Biotechnology (NY) 1991 July;9(7): 630-4): In addition, diabetic retinopathy, macular degeneration based on neovascularization, psoriasis or atherosclerosis (Cherrington J M, Strawn L M, Shawver L K New paradigms for the treatment of cancer, the role of anti-angiogenesis agents, Adv Cancer Res. 2000;79:1-38), ulcerative colitis (Thorn M, Raab Y, Larsson A, Gerdin B, Haligren R. Intestinal mucosal secretion of basic fibroblast growth factor in patients with ulcerative colitis, Scand J Gastroenterol. 2000 April;35(4):408-12) or inflammatory diseases of the colon such as Crohn's disease (Bousvaros A, Leichtner A, Zurakowski D, Kwon J, Law T, Keough K, Fishman S. Elevated serum vascular endothelial growth factor in children and young adults with Crohn's disease, Dig Dis Sci. 1999 February;44(2):424-30). Angiogenesis also plays an important role in cancer (Papac R J, Spontaneous regression of cancer, possible mechanisms. In Vivo. 1998 November-December;12(6):571-8; Giavazzi R, Taraboletti G. Angiogenesis and angiogenesis inhibitors in cancer. Forum (Genoa), 1999 July-September;9(3):261-72). Here, oxygen and nutrients are supplied to the tumor cells by the formation of blood vessels in the growing tumor tissue; likewise a system of conduction is produced to a certain extent, by means of which tumor cells can metastasize throughout the body.

The participation of angiogenesis in such different diseases as cancer, eye diseases and inflammatory diseases has led to the development of methods which are particularly concerned with the inhibition of angiogenesis. For cancer patients, such methods represent a considerable advantage in comparison to conventional methods, such as, e.g., chemotherapy with its massive side effects, which result in part in an unacceptable morbidity or lead to the death of the patient. In practice, these undesired side effects, which are associated with cancer therapies, often limit the treatment, which could help a patient.

For other pathological conditions, which are associated with abnormal angiogenesis, such as, e.g., diabetic retinopathy, there are no effective treatments except for retinal transplants. However, if a retina is transplanted, the new retina would be subjected to the same conditions as the original retina. This makes clear the necessity of identifying molecular interactions which are linked with the angiogenesis that occurs in certain pathological conditions, in order to develop methods for diagnosis and special therapies. Earlier, two main approaches have been followed in principle for the detection and inhibition of pathogenic angiogenesis: first, the inhibition of the process of angiogenesis and vessel arrangement (anti-antiangiogensis*) and secondly, the direct limitation and disruption of the formation of tumor vessels (vascular targeting) Giavazzi R, Taraboletti G. Angiogenesis and angiogenesis inhibitors in cancer, Forum (Genoa), 1999 July-September;9(3):261-72). *sic-Trans. Note.

Investigations that have been conducted recently have made it clear that a genetic switch regulates angiogenesis (Hanahan D, Folkman J., Patterns and emerging mechanisms of the angiogenic switch during tumorigenesis, Cell. 1996 Aug. 9;86(3):353-64). Oncogenes, tumor-suppressive or mutated forms of mitogenic signal metabolic pathways and switch mechanisms in the control of the cell cycle probably regulate genes that are involved in angiogenesis (Folkman J., Angiogenesis in cancer, vascular, rheumatoid and other disease. Nat Med. 1995 January; 1(1):27-31). Of course, the detailed genetic changes and their effects on angiogenesis have still not been identified. The following are viewed as putative candidate genes for angiogenesis, for example: vascular endothelial growth factors (VEGFs) (Ohta Y, Shridhar V, Bright R K, Kalemkerian G P, Du W, Carbone M, Watanabe Y, Pass H I., VEGF and VEGF type C play an important role in angiogenesis and lymphangiogenesis in human malignant mesothelioma tumors., Br J Cancer., 1999 September;81(1):54-61), angiotensins (Machado R D, Santos R A, Andrade S P. Opposing actions of angiotensins on angiogenesis. Life Sci. 2000;66(1):67-76), interleukins (Anderson I C, Mari S E, Broderick R J, Mari B P, Shipp M A, The angiogenic factor interleukin 8 is induced in non-small cell lung cancer/pulmonary fibroblast cocultures, Cancer Res. 2000 Jan. 15;60(2):269-72), or fibroblast growth factors (FGFs) (Dikov M M, Reich M B, Dworkin L, Thomas J W, Miller G G., A functional fibroblast growth factor-1 immunoglobulin fusion protein. J Biol Chem. 1998 Jun. 19;273(25):15811-7).

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized. The prior art which concerns sensitivity is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek A, Oswald J, Walter J. A modified and improved method for bisulphite based cytosine methylation analysis. Nucleic Acids Res. 1996 Dec. 15;24(24):5064-6). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein, T., DePamphilis, M. L., Zorbas, H., Nucleic Acids Res. 1998, 26, 2255.

The bisulfite technique has been previously applied only in research, with a few exceptions (e.g., Zeschnigk M, Lich C, Buiting K, Doerfler W, Horsthemke B. A single-tube PCR test for the diagnosis of Angelman and Prader-Willi syndrome based an allelic methylation differences at the SNRPN locus. Eur J Hum Genet. 1997 March-April;5(2):94-8). However, short, specific segments of a known gene have always been amplified after a bisulfite treatment and either completely sequenced (Olek A, Walter J. The pre-implantation ontogeny of the H19 methylation imprint. Nat Genet. 1997 November;17(3):275-6) or individual cytosine positions have been detected by a “primer extension reaction” (Gonzalgo M L, Jones P A. Rapid quantitation of methylation differences at specific sites using methylation-sensitive single nucleotide primer extension (Ms-SNuPE). Nucleic Acids Res. 1997 Jun. 15;25(12):2529-31, WO-Patent 95-00669) or an enzyme cleavage (Xiong Z, Laird P W., COBRA: a sensitive and quantitative DNA methylation assay. Nucleic Acids Res. 1997 Jun. 15;25(12):2532-4). Detection by hybridization has also been described (Olek et al., WO-A 99-28498).

Other publications which are concerned with the application of the bisulfite technique for the detection of methylation in the case of individual genes are: Grigg G, Clark S. Sequencing 5-methylcytosine residues in genomic DNA. Bioessays. 1994 June;16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March;6(3):387-95; Feil R, Chariton J, Bird A P, Walter J, Reik W. Methylation analysis on individual chromosomes: improved protocol for bisulphite genomic sequencing. Nucleic Acids Res. 1994 Feb. 25;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19;157(1-2):261-4; WO 97-46705, WO 95-15373 and WO 95-45560.

A review of the prior art in oligomer array production can be taken from a special edition of Nature Genetics that appeared in January 1999 (Nature Genetics Supplement, Volume 21, January 1999) and the literature cited therein.

Probes with multiple fluorescent labels have been used for scanning an immobilized DNA array. Particularly suitable for fluorescent labels is the simple introduction of Cy3 and Cy5 dyes at the 5′-OH of the respective probe. The fluorescence of the hybridized probes is detected, for example, by means of a confocal microscope. The dyes Cy3 and Cy5, among many others, are commercially available.

Matrix-assisted laser desorptions/ionization mass spectrometry (MALDI-TOF) is a very powerful development for the analysis of biomolecules (Karas M, Hillenkamp F. Laser desorption ionization of proteins with molecular masses exceeding 10,000 daltons. Anal Chem. 1988 Oct. 15;60(20):2299-301). An analyte is embedded in a light-absorbing matrix. The matrix is vaporized by a short laser pulse and the analyte molecule is transported unfragmented into the gaseous phase. The analyte is ionized by collisions with matrix molecules. An applied voltage accelerates the ions in a field-free flight tube. Ions are accelerated to varying degrees based on their different masses. Smaller ions reach the detector sooner than large ions.

MALDI-TOF spectrometry is excellently suitable for the analysis of peptides and proteins. The analysis of nucleic acids is somewhat more difficult (Gut I G, Beck, S. DNA and Matrix Assisted Laser Desorption Ionization Mass Spectrometry. Current Innovations and Future Trends. 1995, 1; 147-57). For nucleic acids, the sensitivity is approximately 100 times poorer than for peptides and decreases overproportionally with increasing fragment size. For nucleic acids that have a backbone with a multiple negative charge, the ionization process through the matrix is basically less efficient. In MALDI-TOF spectrometry, the choice of matrix plays an imminently important role. Several very powerful matrices, which produce a very fine crystallization, have been found for the desorption of peptides. In the meantime, several effective matrices have been developed for DNA, but the difference in sensitivity was not reduced thereby. The difference in sensitivity can be reduced by modifying the DNA chemically in such a way that it resembles a peptide. Phosphorothioate nucleic acids, in which the usual phosphates of the backbone are substituted by thiophosphates, can be converted by simple alkylation chemistry into a charge-neutral DNA (Gut I G, Beck S. A procedure for selective DNA alkylation and detection by mass spectrometry. Nucleic Acids Res. 1995 Apr. 25;23(8):1367-73). The coupling of a “charge tag” to this modified DNA results in an increase in sensitivity by the same amount as is found for peptides. Another advantage of “charge tagging” is the increased stability of the analysis in the presence of impurities, which make the detection of unmodified substrates very difficult.

Genomic DNA is obtained from DNA of cells, tissue or other test samples by standard methods. This standard methodology is found in references such as Fritsch and Maniatis, eds., Molecular Cloning: A Laboratory Manual, 1989.

Presentation of the Problem

The present invention will provide oligonucleotides and/or PNA oligomers for the detection of cytosine methylations, as well as a method that is particularly suitable for diagnosis and/or therapy of genetic and epigenetic parameters of genes associated with angiogenesis. The invention is based on the knowledge that cytosine methylation patterns are particularly suitable for the diagnosis and/or therapy of diseases associated with angiogenesis.

### DESCRIPTION

The object of the present invention is to provide chemically modified DNA of genes associated with angiogenesis, as well as oligonucleotides and/or PNA oligomers for the detection of cytosine methylations, as well as a method that is particularly suitable for diagnosis and/or therapy of genetic and epigenetic parameters of genes associated with angiogenesis. The invention is based on the knowledge that genetic and epigenetic parameters and particularly cytosine methylation patterns of genes associated with angiogenesis are particularly suitable for the diagnosis and/or therapy of diseases associated with angiogenesis.

This object is solved according to the invention by a nucleic acid comprising a sequence segment that is at least 18 bases long of chemically pretreated DNA of genes associated with angiogenesis according to one of the sequences Seq. ID No. 1 to Seq. ID No. 208 and sequences complementary to these and/or oligonucleotides and/or PNA oligomers according to Table 1. The database numbers (Accession numbers), which define the respective gene sequences as unique, are listed each time behind the indicated gene designations in the Table. GenBank was used as the basic database.

The chemically modified nucleic acids could not previously be related to the determination of genetic and epigenetic parameters.

The object of the present invention is also solved by an oligonucleotide or oligomer for the detection of the cytosine methylation state in chemically pretreated DNA, comprising at least one base sequence with a length of at least 13 nucleotides, which hybridizes to a chemically pretreated DNA of genes associated with angiogenesis according to one of the sequences Seq. ID No. 1 to Seq. ID No. 208 and to sequences complementary to these and/or oligonucleotides and/or PNA oligomers according to Table 1. The oligomer probes according to the invention represent important and effective tools, which make possible for the first time the determination of genetic and epigenetic parameters of genes associated with angiogenesis. Most preferably, the base sequence of the oligomers contains at least one CpG dinucleotide. The probes can also be present in the form of a PNA (peptide nucleic acid), which has particularly preferred pairing properties. Oligonucleotides are particularly preferred according to the invention, in which the cytosine of the CpG dinucleotide is the 5^{th} to the 9^{th} nucleotide from the 5′ end of the 13-mer; in the case of PNA oligomers. it is preferred that the cytosine of the CpG dinucleotide is the 4^{th} to the 6^{th} nucleotide from the 5′ end of the 9-mer.

The oligomers according to the invention are normally used in so-called sets, which comprise at least one oligomer for each of the CpG dinucleotides of one of the sequences Seq. ID No. 1 to Seq. ID No. 208 and sequences complementary to these and/or oligonucleotides and/or PNA oligomers according to Table 1. A set is preferred, which comprises at least one oligomer for each of the CpG dinucleotides of one of Seq. ID No. 1 to Seq. ID No. 208 and sequences complementary to these and/or oligonucleotides and/or PNA oligomers according to Table 1.

In addition, the invention provides a set of at least two oligonucleotides, which can be utilized as so-called primer oligonucleotides for the amplification of DNA sequences of one of Seq. ID No. 1 to Seq. ID No. 208 and sequences complementary to these and/or oligonucleotides and/or PNA oligomers according to Table 1 or segments thereof.

In the case of the set of oligonucleotides according to the invention, it is preferred that at least one oligonucleotide is bound to a solid phase.

The present invention also concerns a set of at least 10 n (oligonucleotides and/or PNA oligomers), which serve for the detection of the cytosine methylation state in chemically pretreated genomic DNA (Seq. ID No. 1 to Seq. ID No. 208 and sequences complementary to these and/or oligonucleotides and/or PNA oligomers according to Table 1). The diagnosis and/or therapy of genetic and epigenetic parameters of genes associated with angiogenesis is possible with these probes. The set of oligomers can also be used for the detection of single nucleotide polymorphisms (SNPs) in the chemically pretreated DNA of genes associated with angiogenesis according to one of the sequences Seq. ID No. 1 to Seq. ID No. 208 and sequences complementary to these and/or oligonucleotides and/or PNA oligomers according to Table 1.

It is preferred according to the invention that an arrangement provided by the invention comprised of different oligonucleotides and/or PNA oligomers (a so-called “array”), is also present bound to a solid phase. This array of different oligonucleotide and/or PNA oligomer sequences can be characterized in that it is arranged on the solid phase in the form of a rectangular or hexagonal grid. Most preferably, the solid-phase surface is comprised of silicon, glass, polystyrene, aluminum, steel, iron, copper, nickel, silver, or gold. Also possible, however, is nitrocellulose as well as plastics, such as nylon, for example, which can be present in the form of spheres or also as a resin matrix.

Another subject of the invention is thus a method for the production of an array fixed onto a support material for analysis in connection with diseases associated with angiogenesis, in which at least one oligomer according to the invention is coupled to a solid phase. Methods for the production of such arrays are known, for example, from U.S. Pat. No. 5,744,305 employing solid-state chemistry and photolabile protective groups.

Another subject of the invention concerns a DNA chip for analysis in connection with diseases associated with angiogenesis, which comprises at least one nucleic acid according to the present invention. DNA chips are known, for example, from U.S. Pat. No. 5,837,832.

The subject of the present invention is also a kit, which can comprise, for example, a reagent containing bisulfite, a set of primer oligonucleotides comprising at least two oligonucleotides, whose sequences correspond to a segment that is at least 18 base pairs long each time of the base sequences listed in the Appendix (Seq. ID No. 1 to Seq. ID No. 208 and to their complementary sequences and/or oligonucleotides and/or PNA oligomers, according to Table 1) or are complementary to them, oligonucleotides and/or PNA oligomers, as well as instructions for conducting and evaluating the described method. A kit in the sense of the invention, however, also may contain only parts of the above-named components.

The invention also provides a method for the determination of genetic and/or epigenetic parameters of genes associated with angiogenesis by analysis of cytosine methylations and single nucleotide polymorphisms, which comprises the following steps:

In a first step of the method, a genomic DNA sample is chemically treated in such a way that cytosine bases that are unmethylated at the 5-position are converted to uracil, thymine or another base unlike cytosine in its hybridization behavior. This is understood in the following as chemical pretreatment.

The genomic DNA to be analyzed is obtained preferably from the usual sources for DNA, such as cells or cell components, for example, cell lines, biopsies, blood, sputum, stool, urine, cerebrospinal fluid, tissue embedded in paraffin, for example, tissue from eyes, intestine, kidney, brain, heart, prostate, lung, breast or liver, histological slides or combinations thereof.

The above-described treatment of genomic DNA with bisulfite (hydrogen sulfite, disulfite) and subsequent alkaline hydrolysis is preferably used for this purpose, which leads to a conversion of unmethylated cytosine nucleobases to uracil or another base unlike cytosine in its base-pairing behavior.

Fragments of this chemically pretreated genomic DNA are amplified with the use of sets of primer oligonucleotides according to the invention and a polymerase, which is most preferably heat-stable. For statistical and practical considerations, it is most preferred that more than ten different fragments, which are 100-2000 base pairs long, are amplified. The amplification of several DNA segments may be conducted simultaneously in one and the same reaction vessel. Usually, the amplification is conducted by means of the polymerase chain reaction (PCR).

In a preferred embodiment of the method, the set of primer oligonucleotides comprises at least two oligonucleotides, whose sequences are each inversely complementary or identical to a segment at least 18 base pairs in length of the base sequences listed in the Appendix (Seq. ID No. 1 to Seq. ID No. 208 and sequences complementary to these and/or oligonucleotides and/or PNA oligomers according to Table 1). The primer oligonucleotides are preferably characterized in that they do not contain a CpG dinucleotide.

It is preferred according to the invention that at least one primer oligonucleotide is bound to a solid phase in the amplification. The different oligonucleotide and/or PNA oligomer sequences can be arranged on a planar solid phase in the form of a rectangular or hexagonal grid, wherein the solid-phase surface is preferably comprised of silicon, glass, polystyrene, aluminum, steel, iron, copper, nickel, silver or gold, whereby other materials such as nitrocellulose or plastics may also be used.

The fragments obtained by means of the amplification can bear a label that can be detected directly or indirectly. Labels are preferred in the form of fluorescent labels, radionuclides, or removable molecular fragments with typical mass, which can be detected in a mass spectrometer, whereby it is preferred that the produced fragments have a single positive or single negative net charge for better detectability in the mass spectrometer. The detection can be carried out and visualized by means of matrix-assisted laser desorption/ionization mass spectrometry (MALDI) or by means of electrospray mass spectrometry (ESI).

The amplificates obtained in the second step of the method are then hybridized to a set of oligonucleotides and/or PNA probes or to an array. Hybridization is then conducted in the manner indicated below. The set used in the hybridization is most preferably comprised of at least 10 oligonucleotide or PNA oligomer probes. The amplificates thus serve as probes, which hybridize to the oligonucleotides previously bound to a solid phase. The unhybridized fragments are then removed. Said oligonucleotides comprise at least one base sequence with a length of 13 nucleotides, which is inversely complementary or identical to a segment of the base sequences listed in the Appendix, which contains at least one CpG dinucleotide. The cytosine of the CpG dinucleotide is the 5th to 9th nucleotide viewed from the 5′-end of the 13-mer. One oligonucleotide is present for each CpG dinucleotide. Said PNA oligomers comprise at least one base sequence with a length of 9 nucleotides, which is inversely complementary or identical to a segment of the base sequences listed in the Appendix, which contains at least one CpG dinucleotide. The cytosine of the CpG dinucleotide is the 4th to 6th nucleotide viewed from the 5′-end of the 9-mer. One oligonucleotide* is present for each CpG dinucleotide. *sic; PNA oligomer?—Trans. Note.

In the fourth step of the method, the unhybridized amplificates are removed.

In the last step of the method, the hybridized amplificates are detected. It is thus preferred that labels that are introduced on the amplificates at each position of the solid phase where an oligonucleotide sequence is found can be identified.

It is preferred according to the invention that the labels of the amplificates are fluorescent labels, radionuclides, or removable molecular fragments with typical mass which can be detected in a mass spectrometer. The amplificates, fragments of the amplificates or probes that are complementary to the amplificates are preferably detected in the mass spectrometer, whereby the detection is carried out and visualized by means of matrix-assisted laser desorption/ionization mass spectrometry (MALDI) or by means of electrospray mass spectrometry (ESI).

The produced fragments can have a single positive or single negative net charge for better detectability in the mass spectrometer. The above-named method for determining genetic and/or epigenetic parameters of genes associated with angiogenesis is preferably used.

The oligomers or arrays of the same according to the invention as well as a kit according to the invention will be used for the diagnosis and/or therapy of diseases associated with angiogenesis, by analysis of methylation patterns of genes associated with angiogenesis. The method is preferably used according to the invention for the diagnosis and/or therapy of important genetic and/or epigenetic parameters within genes associated with angiogenesis.

The method according to the invention serves, for example, for the diagnosis and/or therapy of eye diseases, proliferative retinopathy, neovascular glaucoma, solid tumors, tissue inflammations, rheumatoid arthritis, diabetic retinopathy, macular degeneration based on neovascularization, psoriasis, atherosclerosis, inflammatory intestinal diseases, ulcerative enteritis, Crohn's disease and cancer disorders.

Also, the nucleic acids according to the invention of Seq. ID No. 1 to Seq. ID No. 208 and to sequences complementary to them and/or oligonucleotides and/or PNA oligomers according to Table 1 can be used for the diagnosis and/or therapy of genetic and/or epigenetic parameters of genes associated with angiogenesis.

The present invention also concerns a method for the production of a diagnostic agent or a therapeutic agent for the diagnosis and/or therapy of diseases associated with angiogenesis, by analysis of methylation patterns of genes associated with angiogenesis, whereby the diagnostic agent and/or therapeutic agent is characterized by the fact that at least one nucleic acid according to the present invention, optionally together with suitable additives and adjuvants, is used for its production.

Another subject of the present invention concerns a diagnostic agent and/or therapeutic agent for diseases associated with angiogenesis, by analysis of methylation patterns of genes associated with angiogenesis, which comprises at least one nucleic acid according to the invention, optionally together with suitable additives and adjuvants.

The present invention also concerns the diagnosis and/or prognosis of adverse events for patients or individuals, in which the important genetic and/or epigenetic parameters within genes associated with angiogenesis, which are obtained by means of the invention, can be compared with another set of genetic and/or epigenetic parameters, and the differences thus found serve as a basis for a diagnosis and/or prognosis of adverse events for patients or individuals.

In the sense of the present invention, the term “hybridization” is to be understood as a binding of an oligonucleotide to a completely complementary sequence in the sense of Watson-Crick base pairing with the formation of a duplex structure in the sample DNA. “Stringent hybridization conditions” are understood as those conditions, in which a hybridization occurs at 60° C. in 2.5×SSC buffer, followed by several washing steps at 37° C. in a lower buffer concentration and remains stable.

The term “functional variants” denote all DNA sequences which are complementary to a DNA sequence, which hybridize under stringent conditions with the reference sequence, and have an activity similar to the corresponding polypeptide according to the invention.

“Genetic parameters” in the sense of this invention are mutations and polymorphisms of genes associated with angiogenesis and also to sequences necessary for their regulation. Particularly designated as mutations are insertions, deletions, point mutations, inversions and polymorphisms, and most preferably SNPs (single nucleotide polymorphisms). Polymorphisms, however, can also be insertions, deletions or inversions.

“Epigenetic parameters” in the sense of this invention are particularly cytosine methylations and additional chemical modifications of DNA bases of genes associated with angiogenesis and also to sequences necessary for their regulation. Other epigenetic parameters, for example, are the acetylation of histones, although this cannot be directly analyzed with the described method; however, it is correlated in turn with DNA methylation.

TABLE 1

| Gene name | Accession number |
|---|---|
| ALK-1 | AH005451 |
| Beta-Tubulin | J00314 |
| CCM1 | AF198881 |
| CD44 | AJ251595 |
| ERK | D31661 |
| FGF6 | X57075 |
| FGF receptor-1 | M34641 |
| FLT | E13256 |
| GM-CSF | AJ224149 |
| Platelet glycoprotein IV | AW613186 |
| Gro-alpha | M65005 |
| H-ras | S64238 |
| HGF | E02921 |
| Human interleukin 1-beta IL1B | M15330 |
| ICAM-1 | X84737 |
| ID3 | X73428 |
| IFN-gamma | J00219 |
| IGF-1a | X56773 |
| IGF-1b | X56774 |
| IGF-I | X57025 |
| IL-10 | X73536 |
| IL-18 | E17135 |
| IL-1 | E00846 |
| IL-6 | E02772 |
| KDR | AF063658 |
| KET | Y16961 |
| LPA | U92642 |
| LTBP-1L | AF171934 |
| LTBP-3 | AF011407 |
| M6P IGF2R | S80783 |
| MMP-2 | U96098 |
| MRP-1 | X60111 |
| PDGF beta | AF169594 |
| PTTG2 | AF095288 |
| PTTG | AF062649 |
| Prorenin | E01074 |
| TNF-alpha | X02910 |
| TNF | E03416 |
| Telomerase | AF047386 |
| Tie-2 | W46173 |
| Type IV collagen | U47004 |
| VPF | AA367822 |
| Angiopoietin-4 ANG4 | AF113708 |
| Angiotensinogen II type-1A receptor | M91464 |
| Beta-2 integrin alphaD subunit ITGAD | U40274 |
| eNOS | AF032908 |
| Elongin A | L47345 |
| Endothelial nitric oxide synthase | E16114 |
| fgfr-1 | S76658 |
| Fibronectin | X02761 |
| Gelatinase B | S83357 |
| Heparan sulfate proteoglycan core protein | J04621 |
| Integrin alpha 5 beta 1 | U48214 |
| Integrin alpha 8 subunit | L36531 |
| Integrin alpha 9 | L24158 |
| Interleukin-1 | E00909 |
| Interleukin-2 IL-2 | X00695 |
| Interleukin-6 | M14584 |
| Interleukin-6 receptor | E12979 |
| Interleukin 1 alpha | E04022 |
| Interleukin 1 beta | S61784 |
| Lamin A | L12401 |
| mac25 | L19182 |
| Matrilysin MMP7 | L22524 |
| p15 | S75756 |
| p16 | U12818 |
| CDKN2A | NM 000077 |
| Plasminogen activator inhibitor-1 PAI-1 | M16006 |
| Prostaglandin E2 receptor | L25124 |

The invention will now be further clarified in the following on the basis of sequences and examples, without limiting the invention thereby.

Sequences with odd sequence numbers (e.g., Seq. ID Nos. 1, 3, 5, . . . ) show different sequences each time of the chemically pretreated genomic DNAs of genes associated with angiogenesis.

Sequences with even sequence numbers (e.g., Seq. ID Nos. 2, 4, 6, . . . ) show each time the sequences complementary to the different sequences (e.g., the sequence Seq. ID No. 2 is complementary to Seq. ID No. 1, the sequence Seq. ID No. 4 is complementary to Seq. ID No. 3, and so forth) of the chemically pretreated genomic DNAs of genes associated with angiogenesis.

The following example refers to a fragment of a gene associated with angiogenesis, here TIMP-3 in which a specific CG position will be investigated for its methylation state.

### EXAMPLE 1

### Conducting the Methylation Analysis in the Angiogenesis-Associated Gene, TIMP-3

The following example concerns a fragment of the gene metalloproteinase-3* (TIMP-3), in which a specific CG position is investigated for methylation. *sic; tissue inhibitor of metalloproteinase-3?—Trans. Note.

In the first step, a genomic sequence is treated with the use of bisulfite (hydrogen sulfite, disulfite) in such a way that all of the unmethylated cytosines at the 5-position of the base are modified such that a base that is different in its base-pairing behavior is formed, while the cytosines that are methylated in the 5-position remain unchanged. If bisulfite in the concentration range between 0.1 M and 6 M is used for the reaction, then an addition occurs at the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the detection of methylated cytosines. In the second step of the method, the treated DNA sample is diluted with water or an aqueous solution. A desulfonation of the DNA (10-30 min, 90-100° C.) at alkaline pH is then preferably conducted. In the third step of the method, the DNA sample is amplified in a polymerase chain reaction, preferably with a heat-stable DNA polymerase. In the present case, cytosines of the TIMP-3 gene are investigated. For this purpose, a defined fragment with a length of 401 bp is amplified with the specific primer oligonucleotides AGAGAAATTGGAGGGGTAGT and CCCTCAAACCAATMCAAAA. This amplificate serves as the sample, which hybridizes to an oligonucleotide for example, GGATTTAGCGGTAAGTAT which has previously been bound to a solid phase, with the formation of a duplex structure, whereby the cytosine to be detected is found at position 223 of the amplificate. The detection of the hybridization product is based on primer oligonucleotides fluorescently labeled with Cy3 and Cy5, which were used for the amplification. A hybridization reaction of the amplified DNA with the oligonucleotide occurs only if a methylated cytosine was present at this site in the bisulfite-treated DNA. Thus, the methylation state of the respective cytosine to be investigated decides the hybridization product.

### EXAMPLE 2

### Diagnosis of Diseases Associated With Angiogenesis

In order to be able to relate the methylation pattern to one of the diseases associated with angiogenesis, it is first required that the DNA methylation patterns are investigated for a group of patients with disease and a group of healthy subjects. These investigations are conducted, for example, analogously to Example 1. The results thus obtained are stored in a database and CpG dinucleotides are identified which are differently methylated in the two groups. This can be done by determination of individual CpG methylation rates, as is possible but is conducted relatively inaccurately, e.g., by sequencing, or, however, it can be produced by a very accurate methylation-sensitive “primer extension reaction”. Also, simultaneous analysis of the entire methylation state is possible, and the patterns can be compared, e.g., by means of cluster analyses that can be conducted, e.g., by a computer.

Subsequently, it is possible to assign investigated patients to a specific therapy group and to treat these patients in a targeted manner with an individualized therapy.

Example 2 can be conducted, for example, for the following diseases: eye diseases, proliferative retinopathy, neovascular glaucoma, solid tumors, rheumatoid arthritis, diabetic retinopathy, macular degeneration based on neovascularization, psoriasis, atherosclerosis, ulcerative enteritis, Crohn's disease and cancer disorders.

### EXAMPLE 3

### Conducting Methylation Analysis in the CDKN2A gene

In the first step, a genomic sequence is treated with the use of bisulfite (hydrogen sulfite, disulfite) in such a way that all of the unmethylated cytosines at the 5-position of the base are modified such that a base that is different in its base-pairing behavior is formed, while the cytosines that are methylated in the 5-position remain unchanged. If bisulfite is used for the reaction, then an addition occurs on the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the detection of methylated cytosines. In the second step of the method, the treated DNA sample is diluted with water or an aqueous solution. Preferably, a desulfonation of the DNA is then conducted. In the third step of the method, the DNA sample is amplified in a polymerase chain reaction, preferably with a heat-stable DNA polymerase. The PCR reactions were conducted in a thermocycler (Eppendorf GmbH). For a 25 μl batch, the following were used: 10 ng of DNA, 0.08 μM of each primer oligonucleotide, 1.6 mM dNTPs and one unit of HotstarTaq. The other conditions were selected according to the manufacturer's instructions. For the PCR, first a denaturation was conducted for 15 minutes at 96° C., then 40 cycles (60 seconds at 96° C., 45 seconds at 55° C. and 75 seconds at 65° C.) and a final elongation of 10 minutes at 72° C. The presence of the PCR products was confirmed on agarose gels.

In the present case, cytosines from the potential promotor region of the CDKN2A gene are investigated. With sequences of this gene, samples from patients with the diagnosis of epithelial squamous cell carcinoma of the lungs can be differentiated from samples of the corresponding surrounding normal tissue. For this purpose, a defined fragment with a length of 256 bp is amplified with the specific primer oligonucleotides GGGGTTGGTTGGTTATTAGA (Seq. ID 209) and AACCCTCTACCCACCTAAAT (Seq. ID 210). This amplificate serves as the sample, which hybridizes to an oligonucleotide that has been previously bound to a solid phase, with the formation of a duplex structure, for example, [the oligonucleotide] GGAGTTTTCGGTTGATTG (Seq. ID 211) or GGAGTTTTTGGTTGATTG (Seq. ID 212), whereby the cytosine to be detected is found at position 127 of the amplificate. The methylated cytosine is detected with the oligonucleotide (Seq. ID 211), while, in contrast, the unmethylated state, which is represented by a thymine, is detected with the oligonucleotide (Seq. ID 212). Both oligonucleotides hybridize to the complementary strand each time. The detection of the hybridization product is based on primer oligonucleotides fluorescently labeled with Cy5, which were used for the amplification. A hybridization reaction of the amplified DNA with the oligonucleotide occurs only if a methylated cytosine was present at this site in the bisulfite-treated DNA. Thus the methylation state of the respective cytosine to be investigated decides the hybridization product.

### EXAMPLE 4

### Digital Phenotype

The following example describes the comparison of squamous cell carcinomas of the lungs with the corresponding surrounding normal tissue. Fluorescently labeled primers were used for the multiplex PCRs in order to amplify 8 fragments per reaction. All PCR products from each individual were mixed and hybridized on glass slides on which was applied a pair of immobilized oligonucleotides at each position. Each of these detection oligonucleotides was designed to hybridize to bisulfite-converted sequences found at CpG sites that had been present originally in either the unmethylated (TG) or methylated (CG) state. The hybridization conditions were selected for the detection of differences in single nucleotides of TG and CG variants. The ratios of the two signals were calculated on the basis of a comparison of the intensities of the fluorescing signals.

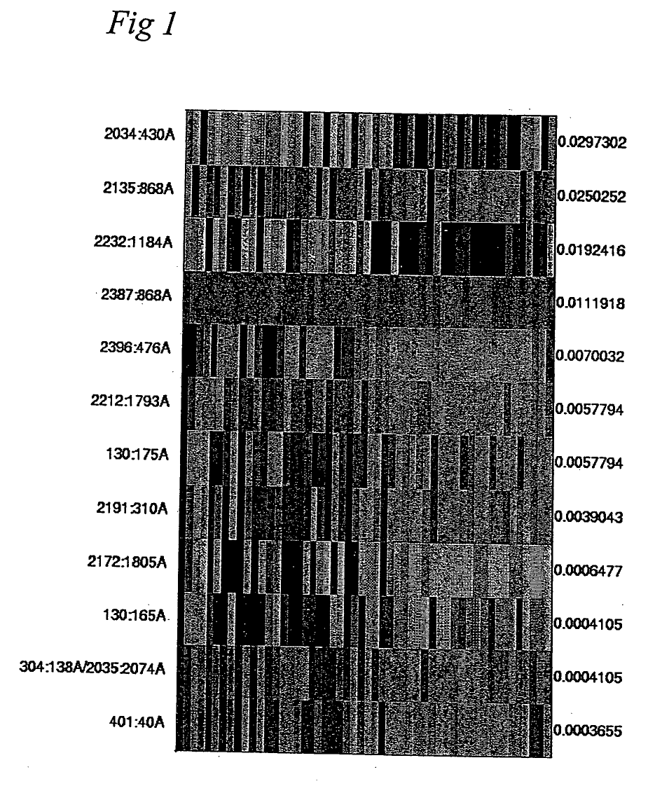

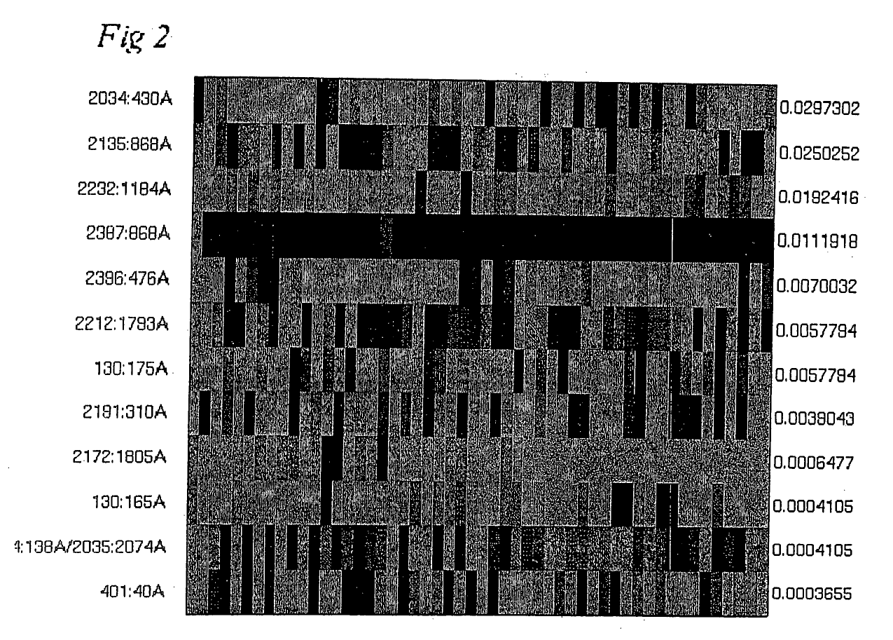

The information is then used in a weighted matrix (see FIG. 1 or 2) to determine the CpG methylation differences between the two classes of tissues. The most significant CpG positions are represented at the lower end of the matrix, and significance decreases toward the top. In FIG. 1, the dark color (red in the original FIG. 2) indicates a high degree of methylation; in FIG. 1, the light color (green in the original FIG. 2) indicates a low degree; and gray in FIG. 1 (dark-gray in the original FIG. 2) indicates an intermediate degree of methylation. Each row represents a specific CpG position in one gene and each column shows the methylation profile of different CpGs for a sample. A gene identification number and a CpG are shown on the left side; the gene name belonging thereto is found in Table 2 each time. The corresponding Accession Numbers of the genes are also listed in Table 2. The number in front of the colon designates the gene name and the number in back of the colon denotes the specific oligonucleotide. The p values of individual CpG positions are shown on the right side of FIG. 1 or 2. The p values represent the probabilities that the observed distribution is random or not.

The first group (on the left in FIG. 1 or 2) contains 26 samples from both sexes, while the second group also contains 26 samples (on the right side in FIG. 1 or 2). The p value of weighted methylation shows a clear distinction between the two groups; 12 CpG positions (red or green color shading) of 11 different genes are significantly different (corrected p value of <0.05) between the two groups. The cross-validated accuracy of the classification, which is determined by SVM (support vector machine) (F. Model, P. Adorjan, A. Olek, C. Piepenbrock, Feature selection for DNA methylation based cancer classification. Bioinformatics. 2001 June;17 Suppl. 1, pp. 157-64), is calculated as 77.0% with a standard deviation of 4.4%. The CDKN2A gene, which is represented by the gene identification number 2035, was investigated here within the scope of a larger study.

TABLE 2

| Gene Number | Gene Name | Accession Number |
|---|---|---|
| 401 | HLA-F | NM 018950 |
| 2035 | CDKN2A | NM 000077 |
| 130 | GPIbbeta | NM 000407 |
| 2172 | MYC | NM 002467 |
| 2191 | PAX6 | NM 001604 |
| 2212 | RARB | NM 000965 |
| 2396 | TMEFF2 (=HPP1) | NM 016192 |
| 2387 | PTGS2 | NM 000963 |
| 2232 | SFN | NM 006142 |
| 2135 | LKB1 | NM 000455 |
| 2034 | CDKN1B | NM 004064 |

Description of FIG. 1

Differentiating squamous cell carcinomas of the lungs from the corresponding surrounding normal tissue. Dark signals correspond to a high probability for methylation, light signals correspond to a low probability, and gray shades to intermediate values. The 26 samples on the left side of FIG. 1 are assigned to the squamous cell carcinomas, while the 26 samples on the right side correspond to the normal tissue.

Description of FIG. 2

This figure corresponds to FIG. 1, but reference is made here to the filed original figure (i.e., color figure); the figure is reproduced according to the manufacturer's instructions (Axon Instruments, Gene Pix 4000A, Program Version: GenePixPro 3.0.6.66). Differentiating squamous cell carcinomas of the lungs from the corresponding surrounding normal tissue. Red signals correspond to a high probability for methylation, green signals correspond to a low probability, and gray shades to intermediate values. The 26 samples on the left side of FIG. 2 are assigned to the squamous cell carcinomas, while the 26 samples on the right side correspond to the normal tissue.

1. Nucleic acids comprising a sequence segment at least 18 bases long of the chemically pretreated DNA of genes associated with angiogenesis according to one of the sequences Seq. ID 1 to Seq. ID 208 and to their complementary sequences.

2. An oligomer (oligonucleotide or peptide nucleic acid (PNA) oligomer) for the detection of the cytosine methylation state in chemically pretreated DNA, each comprising at least one base sequence with a length of at least 9 nucleotides, which hybridizes to a chemically pretreated DNA of genes associated with angiogenesis according to one of the sequences Seq. ID No. 1 to Seq ID No. 208 and to their complementary sequences.

3. The oligomer according to claim 2, whereby the base sequence comprises at least one CpG dinucleotide.

4. The oligomer according to claim 3, further characterized in that the cytosine of the CpG dinucleotide is found in approximately the middle third of the oligomer.

5. Oligonucleotides and/or PNA oligomers for the detection of the cytosine methylation state in chemically pretreated DNA, each comprising at least one base sequence with a length of at least 9 nucleotides, which hybridizes to a chemically pretreated DNA according to one of the following genes associated with angiogenesis: ALK-1 (AH005451), Beta-tubulin (J00314), CCM1 (AF198881), CD44 (AJ251595), ERK (D31661), FGF6 (X57075), FGF receptor-1 (M34641), FLT (E13256), GM-CSF (AJ224149), Platelet glycoprotein IV (AW613186), Gro-alpha (M65005), H-ras (S64238), HGF (E02921), Human interleukin 1-beta IL1B (Ml 5330), ICAM-1 (X84737), ID3 (X73428), IFN-gamma (J00219), IGF-1a (X56773), IGF-1b (X56774), IGF-I (X57025), IL-10 (X73536), IL-18 (E17135), IL-1 (E00846), IL-6 (E02772), KDR (AF063658), KET (Y16961), L18a (L05093), LPA (U92642), LTBP-1L (AF171934), LTBP-3 (AF011407), M6P IGF2R (S80783), MMP-2 (U96098), MRP-1 (X60111), PDGF beta (AF169594), PTTG2 (AF095288), PTTG (AF062649), Prorenin (E01074), TNF-alpha (X02910), TNF (E03416), Telomerase (AF047386), Tie-2 (W46173), Type IV collagen (U47004), VPF (AA367822), Angiopoietin-4 ANG4 (AF113708), Angiotensinogen II type-1A receptor (M91464), Beta-2 integrin alpha-D subunit ITGAD (U40274), eNOS (AF032908), Elongin A (L47345), Endothelial nitric oxide synthase (E16114), fgfr-1 (S76658), Fibronectin (X02761), Gelatinase B (S83357), Heparan sulfate proteoglycan core protein (J04621), Integrin alpha 5 beta 1 (U48214), Integrin alpha 8 subunit (L36531), Integrin alpha 9 (L24158), Interleukin-1 (E00909), Interleukin-2 IL-2 (X00695), Interleukin-6 (M14584), Interleukin-6 receptor (E12979), Interleukin 1 alpha (E04022), Interleukin 1 beta (S61784), Lamin A (L12401), mac25 (L19182), Matrilysin MMP7 (L22524), p15 (S75756), p16 (U12818), CDKN2A (NM_{—}000077), Plasminogen activator inhibitor-1 PAI-1 (M16006) or Prostaglandin E2 receptor (L25124), wherein the base sequence comprises at least one CpG dinucleotide and wherein the oligomer is characterized in that the cytosine of the CpG dinucleotide is found in approximately the middle third of the oligomer.

6. A set of oligomers according to claim 3 or claim 5, comprising at least one oligomer for the detection of the methylation state of at least one of the CpG dinucleotides from one of the sequences of Seq. ID 1 to Seq. ID 208 and sequences complementary to these and/or oligonucleotides and/or PNA oligomers from claim 5.

7. A set of oligomers according to claim 3 or claim 5, comprising oligomers for the detection of the methylation state of all CpG dinucleotides from one of the sequences of Seq. ID 1 to Seq. ID 208 and sequences complementary to these and/or oligonucleotides and/or PNA oligomers from claim 5.

8. A set of at least two oligonucleotides according to claim 2, which can be utilized as primer oligonucleotides for the amplification of DNA sequences of one of the sequences Seq. ID 1 to Seq. ID 208 and sequences complementary to these and/or oligonucleotides and/or PNA oligomers from claim 5 or segments thereof.

9. The set of oligonucleotides according to claim 8, further characterized in that at least one oligonucleotide is bound to a solid phase.

10. A set of oligomer probes for the detection of the cytosine methylation state and/or of single nucleotide polymorphisms (SNPs) in chemically pretreated genomic DNA according to one of the sequences Seq. ID 1 to Seq. ID 208 and to their complementary sequences and/or oligonucleotides and/or PNA oligomers from claim 5, comprising at least ten of the oligomers according to one of claims 2 to 4.

11. A method for the production of an arrangement of different oligomers (array) fixed onto a support material for the analysis of disorders related to the methylation state of the CpG dinucleotides of one of the sequences Seq. ID 1 to Seq. ID 208 and to their complementary sequences and/or oligonucleotides and/or PNA oligomers from claim 5, in which at least one oligomer according to one of claims 2 to 4 is coupled to a solid phase.

12. An arrangement of different oligomers (an array) according to one of claims 2 to 4, which is bound to a solid phase.

13. The array of different oligonucleotide and/or PNA oligomer sequences according to claim 12, further characterized in that these are arranged on a planar solid phase in the form of a rectangular or hexagonal grid.

14. The array according to one of claims 12 or 13, further characterized in that the solid-phase surface is preferably comprised of silicon, glass, polystyrene, aluminum, steel, iron, copper, nickel, silver, or gold.

15. A DNA and/or PNA array for the analysis of disorders related to the methylation state of genes, which contains at least one nucleic acid according to one of the preceding claims.

16. A method for determining genetic and/or epigenetic parameters for the diagnosis and/or therapy of existing diseases or of the predisposition for specific diseases by analysis of cytosine methylations, is hereby characterized in that the following steps are conducted:
a) in a genomic DNA sample, cytosine bases that are unmethylated at the 5-position are converted by chemical treatment to uracil or another base unlike cytosine in its base-pairing behavior;
b) fragments of this chemically pretreated genomic DNA are amplified with the use of sets of primer oligonucleotides according to claim 8 or 9 and a polymerase, whereby the amplificates bear a detectable label;
c) the amplificates are hybridized to a set of oligonucleotides and/or PNA probes according to claims 2 to 4 or, however, to an array according to one of claims 12 to 14;
d) the hybridized amplificates are then detected.

17. The method according to claim 16, further characterized in that the chemical treatment is conducted by means of a solution of a bisulfite, hydrogen sulfite or disulfite.

18. The method according to one of claims 16 or 17, further characterized in that more than ten different fragments, which are 100-2000 base pairs long, are amplified.

19. The method according to one of claims 16 to 18, further characterized in that the amplification of several DNA segments is conducted in one reaction vessel.

20. The method according to one of claims 16 to 19, further characterized in that the polymerase is a heat-stable DNA polymerase.

21. The method according to claim 20, further characterized in that the amplification is conducted by means of the polymerase chain reaction (PCR).

22. The method according to one of claims 16 to 21, further characterized in that the labels of the amplificates are fluorescent labels.

23. The method according to one of claims 16 to 21, further characterized in that the labels of the amplificates are radionuclides.

24. The method according to one of claims 16 to 21, further characterized in that the labels of the amplificates are removable molecular fragments with typical mass, which are detected in a mass spectrometer.

25. The method according to one of claims 16 to 21, further characterized in that the amplificates or fragments of the amplificates are detected in the mass spectrometer.

26. The method according to one of claims 24 and/or 25, further characterized in that the produced fragments have a single positive or single negative net charge for better detectability in the mass spectrometer.

27. The method according to one of claims 24 to 26, further characterized in that the detection is carried out and visualized by means of matrix-assisted laser desorption/ionization mass spectrometry (MALDI) or by means of electrospray mass spectrometry (ESI).

28. The method according to one of claims 16 to 27, further characterized in that the genomic DNA was obtained from cells or cell components that contain DNA, whereby sources of DNA comprise e.g., cell lines, biopsies, blood, sputum, stool, urine, cerebrospinal fluid, tissue embedded in paraffin, for example, tissue from eyes, intestine, kidney, brain, heart, prostate, lung, breast or liver, histological slides and all possible combinations thereof.

29. A kit, comprising a bisulfite (=disulfite, hydrogen sulfite) reagent as well as oligonucleotides and/or PNA oligomers according to one of claims 2 to 4.

30. Use of a nucleic acid according to claim 1, an oligonucleotide or PNA oligomer according to one of claims 2 to 4, a kit according to claim 29, an array according to one of claims 11 to 14, a set of oligonucleotides comprising at least one oligomer for the detection of the methylation state of at least one of the CpG dinucleotides from one of the sequences of Seq. ID 1 to Seq. ID 208 and sequences complementary to these and/or oligonucleotides and/or PNA oligomers from claim 5 for the diagnosis of eye diseases, proliferative retinopathy, neovascular glaucoma, solid tumors, tissue inflammations, rheumatoid arthritis, diabetic retinopathy, macular degeneration based on neovascularization, psoriasis, atherosclerosis, inflammatory intestinal diseases, ulcerative enteritis, Crohn's disease and cancer disorders.

31. Use of a nucleic acid according to claim 1, an oligonucleotide or PNA oligomer according to one of claims 2 to 4, a kit according to claim 29, an array according to one of claims 11 to 14, a set of oligonucleotides comprising at least one oligomer for the detection of the methylation state of at least one of the CpG dinucleotides from one of the sequences of Seq. ID 1 to Seq. ID 208 and sequences complementary to these and/or oligonucleotides and/or PNA oligomers from claim 5 for the therapy of eye diseases, proliferative retinopathy, neovascular glaucoma, solid tumors, tissue inflammations, rheumatoid arthritis, diabetic retinopathy, macular degeneration based on neovascularization, psoriasis, atherosclerosis, inflammatory intestinal diseases, ulcerative enteritis, Crohn's disease and cancer disorders.

32. A kit, comprising a bisulfite (=disulfite, hydrogen sulfite) reagent as well as oligonucleotides and/or PNA oligomers according to claim 31.
